(19) United States
(12) Patent Application Publication
(10) Pub. No.: US 2009/0298109 A1
(43) Pub. Date: Dec. 3, 2009

(54) METHOD FOR IDENTIFYING BPLP AND OPIORPHIN AGONISTS OR ANTAGONISTS

(75) Inventor: Catherine Rougeot, Chevreuse (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: INSTITUT PASTEUR, Paris Cedex (FR)

(21) Appl. No.: 12/357,192

(22) Filed: Jan. 21, 2009

Related U.S. Application Data
(60) Provisional application No. 61/021,988, filed on Jan. 18, 2008.

(51) Int. Cl. C12Q 1/37

(52) U.S. Cl. 435/24

(57) ABSTRACT

A method for in vitro functional characterization of Opiorphin derivatives by using highly selective biochemical assays. The method may employ an assay involving contacting an Opiorphin derivative with an enkephalin-inactivating ectopeptidase, such as neutral endopeptidase NEP (EC 3.4.24.11) or aminopeptidase AP-N (EC 3.4.11.2). This method provides a rapid and sensitive assay for measuring activity of these two membrane-anchored ectoenzymes when contacted with Opiorphin derivative by means of a selective fluorescence-based enzyme model.

### CROSS-REFERENCE TO RELATED APPLICATIONS

This application claims priority to U.S. Provisional Application No. 61/021,088, filed Jan. 18, 2008, which is hereby incorporated by reference in its entirety.

### BACKGROUND OF THE INVENTION

1. Field of the Invention

Methods for identifying and characterizing BPLP and Opiorphin products and their derivatives having BPLP or Opiorphin agonist or antagonist activity.

2. Description of the Related Art

Human Opiorphin is a natural antinociceptive modulator of opioid-dependent pathways. Human Opiorphin native peptide characterized by the sequence QRFSR (SEQ ID NO: 1) has been previously identified as an efficient dual inhibitor of two enkephalin-inactivating ectopeptidases, neutral endopeptidase NEP (EC 3.4.24.11), and aminopeptidase AP-N (EC 3.4.11.2), see Wisner et al. PNAS, November 2006, 103(47): 17979-84 and WO2005/090386.

Mammalian zinc ectopeptidases play important roles in turning off neural and hormonal peptide signals at the cell surface, notably those processing sensory information. Opiorphin displays potent analgesic activity in chemical and mechanical pain models by activating endogenous opioid-dependent transmission. Its function is closely related to the rat sialorphorin peptide, which is an inhibitor of pain perception and acts by potentiating endogenous μ- and δ-opioid receptor-dependent enkephalinergic pathways. The pain-suppressive potency of Opiorphin is as effective as morphine in the behavioral rat model of acute mechanical pain, the pin-pain test; Wisner et al. PNAS, November 2006, 103(47): 17979-84.

Opiorphin (QRFSR (SEQ ID NO: 1)-peptide) is a maturation product of BPLP (basic proline-rich lacrimal protein or PROL1 gene), Wisner, et al., id. The structural and functional characteristics, including the sequence of BPLP (or PRL1) gene, the encoded BPLP polypeptide and its maturation products are described by reference to Wisner, et al., id., Rougeot, et al., Biomed. Rev. 9:17 (1998), Dickenson, et al., Curr. Eye Res. 15:377 (1996) and WO2005/090386, each of which is hereby specifically incorporated by reference as describing the characteristics of BPLP and BPLP maturation products such as Opiorphin.

### BRIEF SUMMARY OF THE INVENTION

The inventors disclose herein methods for identifying compounds that exhibit one or more biochemical or pharmacological properties of native BPLP, its maturation products, or Opiorphin, BPLP. These methods include functional characterization of such derivatives in vitro using highly selective biochemical assays. For example, rapid and sensitive assays for detecting agonist or antagonist activity of Opiorphin derivatives have been developed by measuring the activity of the two membrane-anchored ectoenzymes NEP and APN using a selective fluorescence-based enzyme model.

One aspect of the invention is a method performed in vitro for screening or identifying one or more candidate compounds for their ability to act as agonists or antagonists of BPLP (basic proline-rich lacrimal protein or PROL1 gene product) or maturation products thereof on hNEP or hAP-N activity, which comprises a) incubating a candidate compound with a soluble hNEP (human neutral ecto-peptidase) or hAP-N (human ecto-aminopeptidase), in the presence of a hNEP or hAP-N substrate; b) determining the hydrolysis rate of the NEP or AP-N substrate by the hNEP or hAP-N pure soluble enzyme, wherein an increased hydrolysis rate in the presence of the candidate compound, in comparison with the hydrolysis in the absence of the candidate compound, in initial velocity conditions, is indicative of an antagonist activity; and wherein a decreased hydrolysis rate in the presence of the candidate compound, in comparison with the hydrolysis in the absence of the candidate compound, is indicative of an agonist activity.

Such a method may be used to identify or screen candidate compounds for their ability to act as Opiorphin agonists or antagonists, since Opiorphin is a maturation product of BPLP. The method may be used to identify an agonist or an antagonist of BPLP or a maturation product thereof. It may be conducted in the presence of a NEP substrate or an AP-N substrate. A substrate that is specific for NEP-endopeptidase activity, such as Abz-dR-G-L-EDDnp FRET-peptide may be employed; a substrate that is specific for or which reacts with for NEP-carboxydipeptidase activity, such as Abz-R-G-F-K-DnpOH FRET-peptide or Mca-R-P-P-G-F-S-A-F-K-(Dnp)-OH FRET-peptide (Mca-BK2). A substrate that is specific for aminopeptidase activity, such as L-alanine-Mca (Ala-Mca) may also be used. Any of these substrates may be in the form of a fluorophore-peptide and the method may be performed as real-time fluorescence monitoring microplate adapted fluorimetric assay.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1: The kinetic of Abz-dRGL-EDDnp breakdown by recombinant hNEP in the presence of corresponding vehicle (cross-shaped) or in the presence of 2.5 to 70 μM QRFSR (SEQ ID NO: 1)-peptide. Each point represents the intensity of the signal expressed in RFU (Relative Fluorescence Unit), which was directly proportional to the quantity of metabolites formed, as function of reaction time (min).

FIG. 2: Concentration-dependent inhibition by QRFSR (SEQ ID NO: 1)-peptide of Abz-dRGL-EDDnp breakdown by pure recombinant human hNEP. Each point (open squares) represents the percentage of intact substrate recovered and calculated as: percentage of velocity without inhibitor-velocity in presence of inhibitor/velocity without inhibitor, which was measured in the absence or in the presence of various concentrations of QRFSR (SEQ ID NO: 1)-peptide plotted in μM (log-scale).

FIG. 3: Concentration-dependent inhibition by pGluRFSR native peptide (open squares) of Abz-RGFK-DnpOH breakdown by pure recombinant human hNEP. Each point represents the percentage of intact substrate recovered and calculated as: percentage of velocity without inhibitor-velocity in presence of inhibitor/velocity without inhibitor, which was measured in the absence or in the presence of various concentrations of pGlu-RFSR-peptide plotted in μM (log-scale).

FIG. 4: The kinetic of Ala-AMC breakdown by recombinant hAP-N in absence of inhibitor (black circles) or in the presence of 1 to 60 μM QRFSR (SEQ ID NO: 1)-peptide. Each point represents the intensity of the signal expressed in RFU (Relative Fluorescence Unit), which was directly proportional to the quantity of metabolites formed, as function of reaction time (min).

FIG. 5: Concentration-dependent inhibition by QRFSR (SEQ ID NO: 1)-peptide of Ala-AMC breakdown by pure recombinant human AP-N. Each point (open squares) represents the percentage of intact substrate recovered after incubation and calculated as: percentage of velocity without inhibitor-velocity in presence of inhibitor/velocity without inhibitor, which was measured in the absence or in the presence of various concentrations of QRFSR (SEQ ID NO: 1)-peptide plotted in μM (log-scale).

FIG. 6: The kinetic of Ala-AMC breakdown by recombinant HAP-N in absence of inhibitor (black squares & triangles) or in the presence of 10 to 60 μM pGlu-RFSR peptide. Each point represents the intensity of the signal expressed in RFU (Relative Fluorescence Unit), which was directly proportional to the quantity of metabolites formed, as function of reaction time (min).

FIG. 7: FRET-based enzyme in vitro models: effect of Y[C12-polyethylene spacer]QRFSR (SEQ ID NO: 1) Opiorphin peptide on hNEP & hAP-N activity. A representative enzyme kinetic profile showing the rate of Abz-dR-G-L-EDDnp substrate hydrolysis by recombinant hNEP endopeptidase activity in the presence of vehicle or in the presence of various concentrations of Y[PE12]QRFSR (SEQ ID NO: 2)-peptide analog. Each point represents the intensity of the signal expressed in RFU (Relative Fluorescence Unit), which is proportional to the quantity of metabolites formed, as function of reaction time (min).

FIG. 8: Y[PE12]QRFSR (SEQ ID NO: 2) effect on specific NEPCDP activity. A representative enzyme kinetic profile showing the rate of Abz-R-G-F-K-DnpOH substrate hydrolysis by recombinant hNEP-carboxydipeptidase activity in the presence of vehicle or in the presence of various concentrations of Y[PE12]QRFSR (SEQ ID NO: 2)-peptide analog. Each point represents the intensity of the signal expressed in RFU (Relative Fluorescence Unit), which is proportional to the quantity of metabolites formed, as function of reaction time (min).

FIG. 9: Inhibition of FRET-substrate hydrolysis by human ectopeptidases (%). Concentration-dependent inhibition by Y[PE12]QRFSR (SEQ ID NO: 2) peptide of hydrolysis of the corresponding FRET-peptide substrates by pure recombinant human hNEP or HAP-N. Each point represents the percentage of intact substrate recovered and calculated as: percentage of velocity without inhibitor-velocity in presence of inhibitor/velocity without inhibitor, which was measured in the absence or in the presence of various concentrations of compound plotted in μM (log-scale). Each point is the mean±SD of 3-5 replicates Y[PE12]QRFSR (SEQ ID NO: 2) peptide prevented in a concentration dependent manner the Abz-dR-G-L-EDDnp cleavage mediated by the rhNEP-Endopeptidase activity: r²=0.988, n=38 determination points. The half-maximal inhibitory potency (IC50) was at 8 μM. Y[PE12]QRFSR (SEQ ID NO: 2) peptide prevented in a concentration dependent manner the Abz-R-G-F-K-DnpOH cleavage mediated by the rhNEP-CarboxyDiPeptidase activity: r²=0.964, n=48 determination points. The IC50 was determined at 14 μM Y[PE12]QRFSR (SEQ ID NO: 2) peptide prevented in a concentration dependent manner the Mca-R-P-P-G-F-S-A-F-K-(Dnp)-OH FRET-peptide (Mca-BK2) cleavage mediated by the rhNEP activity: r²=0.966, n=24 determination points respectively. The IC50 was determined at 13 μM. Y[PE12]QRFSR (SEQ ID NO: 2) peptide prevented in a concentration dependent manner the Ala-AMC cleavage mediated by the rhAP-N activity: r²=0.987, n=14 determination points. The IC50 was ≧50 μM. As shown on FIG. 10 the Y-[C12-polyethylene spacer]QRFSR (SEQ ID NO: 1) peptide displayed inhibitory activity towards human NEP-EndoPeptidase activity and NEP-CarboxyDi-Peptidase activity. Its half inhibitory potency (IC50) on hNEP, was evaluated at 8 μM and 13-14 μM respectively; however its inhibitory potency towards human AP-N (IC50≧50 μM) is at least five times weaker than that of the native QRFSR (SEQ ID NO: 1) Opiorphin-peptide. Thus, substituting the terminal NH2 group of the Glutamine by a 12 Carbon-polyethylene spacer plus a Tyrosine residue resulted in strongly decreasing the inhibitory potency of QRFSR (SEQ ID NO: 1) Opiorphin-peptide towards hAP-N but reinforcing its inhibitory potency towards hNEP-endopeptidase and hNEP-carboxypeptidase activities.

### DETAILED DESCRIPTION OF THE INVENTION

As used herein, an agonist of a BPLP protein or maturation product thereof, especially Opiorphin, is a molecule which has the ability to inhibit in a dose-dependent manner a metallo-ectopeptidase activity, especially NEP or APN activity, and therefore to decrease in a dose-dependent manner hydrolysis by said metallo-ectopectidase of its substrate. In particular embodiments, the inhibition of the hydrolysis of the metallo-ectopeptidase substrate obtained with said agonist can be 2 fold less than, preferably similar to (more or less 10%), and more preferably greater than the inhibition of the hydrolysis of the metallo-ectopeptidase substrate obtained with Opiorphin. In a particular embodiment, said agonist has the ability to inhibit in a dose-dependent manner the metallo-ectopeptidase activity of NEP and APN, and therefore to decrease in a dose-dependent manner hydrolysis by NEP and by APN of their respective substrates.

As used herein, an antagonist of a BPLP protein or maturation product thereof, especially Opiorphin, is a molecule which has the ability to increase in a dose-dependent manner a metallo-peptidase activity, especially NEP or APN activity, and therefore to increase in a dose-dependent manner hydrolysis by said metallo-ectopectidase of its substrate.

Preferably, the agonists and antagonists which are identified or screened by the method of the invention are first obtained by structural modification of BPLP or maturation products thereof, especially Opiorphin.

Structural Modification of BPLP or Opiorphin

Modified forms of BPLP and Opiorphin polypeptides, such as polypeptides containing insertions, deletions, or substitutions of amino acid residues, may be produced by methods well known in the art based on the known amino acid and polynucleotide sequences of these products. The BPLP amino acid sequence and the corresponding polynucleotide sequence are incorporated by reference to Dickinson and Thiesse, et al., Curr. Eye Res. 15(4):377-386 (1996) and to SEQ ID NOS: 1 and 2 in WO2005/090386 which respectively depict the BPLP cDNA sequence and the BPLP amino acid sequence. Modified forms of BPLP or Opiorphin include the maturation products, fragments of BPLP (e.g., from 3 to about 100, preferably 3 to 15, consecutive amino acid residues) and peptide derivatives described by pages 7-11 of WO2005/090386 which is incorporated by reference as describing these products as well as methods for making and identifying them. This incorporation by reference also includes each of the scientific documents described on pages 8-11 of WO2005/090386 which refer, for example, methods for making peptide derivatives, mimetics and peptidomimetics. Such derivatives include those conforming to Xaa-Xaa-Arg-Phe-Ser-Arg (SEQ ID NO: 3), where X1 and X2 may be absent or may be any amino acid or modified amino acid residue. Amino acid residues other than the conventional twenty amino acids are contemplated, residues such as pyroGlutamic acid are included. Modified amino acid residues refer to amino acids which can form a peptide bond to at least one other amino acid in the peptide chain, but which may have modified side chains or chemical spacer groups, e.g., Y[PE12]-.

BPLP and Opiorphin products and derivatives may be collected and purified by means well known in the art, including by HPLC chromatography, immunoaffinity techniques, such as antibody-based techniques or other such methods. Preferably, in the methods of the invention, these products will be used in a purified or isolated form which means at least 75 wt. % of the purified product, preferably at least 85, 90, 95, 98, 99 or 100 wt. %.

Substrates for NEP and APN are well-known in the art and are incorporated by reference to Wisner et al. PNAS, November 2006, 103(47): 17979-84 and WO2005/090386. For example, Substance P is an NEP physiological substrate. Specific substrates are also disclosed herein and on pages 40-41 and the Examples of WO 2005/090386 which is hereby incorporated by reference. Peptidase activities may be determined using the methods described on pages 41 and 42 and the Examples of WO 2005/090386 or Wisner, PNAS, id. which are incorporated by reference.

Biochemical Assays: Method & Validation

Real-time fluorescence monitoring of chemical reactions, such as proteolysis, is well-known in the art and such methods are incorporated by reference to Rodems, et al., Assay Drug Dev Technol. 2002 November; 1(1 Pt 1):9-19; Felber, et al., Biotechniques. 2004 May; 36(5):878-85; Matsuo, et al., Microbes Infect. 2006 January; 8(1):84-91. Epub (2005); and Tatham, et al., Methods Mol Biol. 2009; 497:253-68. However, a highly selective, rapid, and convenient biochemical assay useful for the in vitro identification of functional Opiorphin derivatives were unknown and unvalidated for use in conjunction with Opiorphin.

Employing the materials such as those disclosed below, the inventors developed and validated the use of such assays for rapidly detecting compounds that are Opiorphin agonists or antagonists and for quantifying functional differences between Opiorphin activities amongst these compounds. In these assays, formal kinetic analysis was performed using real-time fluorescence monitoring of specific substrate hydrolysis. For each 96-well adapted fluorimetric model, all parameters allowing the analysis of human NEP and human AP-N enzyme activity were defined under conditions of initial velocity measurement.

I. Sources of the human ectopeptidases, hNEP and hAP-N Recombinant human NEP and recombinant human AP-N (devoid of their respective N-terminal cytosol and transmembrane segment) were purchased from R&D Systems (USA) and were used as pure source of peptidase.

II. Substrates and Synthetic Inhibitors

In vitro, amino-, carboxydi- and endo-peptidase activities were assayed by measuring the breakdown of the following synthetic selective substrates:
Abz-dR-G-L-EDDnp FRET-peptide that is an internally quenched fluorescent substrate specific for NEP-endopeptidase activity, was synthesized by Thermo-Fisher Scientific (Germany).
Abz-R-G-F-K-DnpOH FRET-peptide is an internally quenched fluorescent substrate specific for NEP-carboxydipeptidase activity, was synthesized by Thermo-Fisher Scientific (Germany)
Mca-R-P-P-G-F-S-A-F-K-(Dnp)-OH FRET-peptide (Mca-BK2) is an intramolecularly quenched fluorogenic peptide structurally related to bradykinin, which is a selective substrate for measuring NEP and ECE activity, was purchased from R&D Systems.
   FRET is the distance-dependant transfer of energy from a donor fluorophore (Abz=ortho-aminobenzoyl or Mca=7-methoxycoumarin-4-yl-acetyl) to an acceptor fluorophore (DnpOH=2,4-dinitrophenyl or EDDnp=2,4-dinitrophenyl ethylenediamine).

L-alanine-Mca, Ala-Mca, a fluorogenic substrate for measuring aminopeptidase activity was purchased from Sigma.

Measuring the hydrolysis rate of these substrates by soluble ectopeptidases in the presence and absence of different available selective synthetic peptidase inhibitors assessed the specificity of each enzyme assay:—thiorphan (NEP inhibitor) (Bachem),—Bestatin (AP inhibitor) (Calbiochem).

III. Measurement of Ectopeptidase Activities Using 96-Well Adapted Fluorimetric Assays

According to conditions of initial velocity measurement: the time, pH and temperature of incubation as well as enzyme and substrate concentrations were defined for each assay.

Hydrolysis of substrates was measured by real-time monitoring their metabolism rate by the two peptidases in the presence and absence of tested inhibitory compound (concentrations ranging from 1 to 70 μM). These were added to the preincubation medium. The background rate of substrate autolysis representing the fluorescent signal obtained in the absence of enzyme was subtracted to calculate the initial velocities in RFU (Relative Fluorescent Unit)/min.

A. Measurement of NEP-endopeptidase activity using FRETspecific peptide-substrate, Abz-dR-G-L-EDDnp. Using black half-area 96 well microplate, the standard reaction consisted of enzyme (12.5 ng) in 100 mM Tris-HCl pH 7 containing 200 mM NaCl (100 μl final volume). The substrate (15 μM final concentration) was added after preincubation for 10 min at 28° C. and the kinetics of appearance of the fluorescent signal (RFU) was directly analyzed for 40 min at 28° C. (2.3 min-interval successive measures) by using a fluorimeter microplate reader (monochromator Infinite 200-Tecan) at 320 nm and 420 nm excitation and emission wavelengths, respectively.

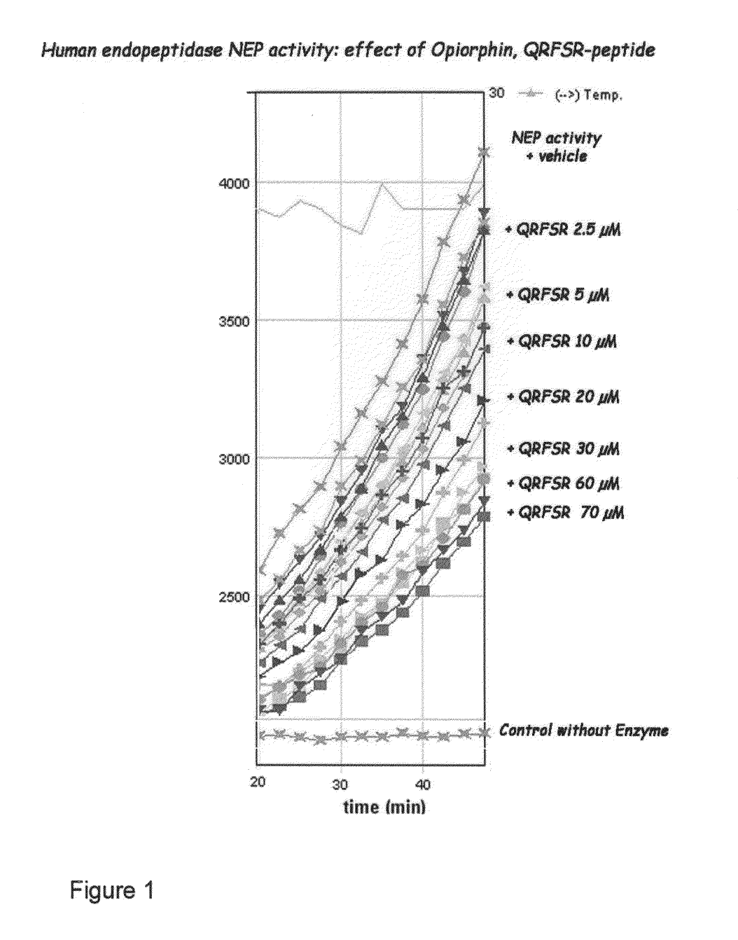

As shown by FIG. 1, under conditions of initial velocity measurement, the intensity of the signal was directly proportional to the quantity of metabolites formed during the 20-40 min time-period of the reaction. Thus, in absence of inhibitor, the initial velocity of rhNEP-mediated specific endoproteolysis of Abz-dR-G-L-EDDnp, was calculated from the linear regression (slope ═NEP activity in presence of vehicle/incubation time, FIG. 1) as 8218±2878 RFU/min/μg rhNEP, n=3 independent determinations.

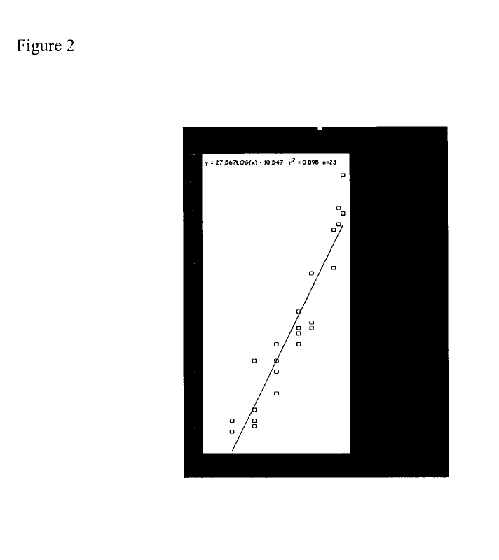

Under these experimental conditions, Opiorphin QRFSR (SEQ ID NO: 1)-peptide prevented in a concentration dependent manner the Abz-dR-G-L-EDDnp cleavage mediated by the rhNEP-Endopeptidase activity (r²=0.90, n=23 determination points, see FIG. 2).

B. Measurement of NEP-carboxydipeptidase activity using FRET specific peptide-substrate Abz-R-G-F-K-DnpOH. Using black half-area 96 well microplate, the standard reaction consisted of enzyme (2.5 ng) in 100 mM Tris-HCl pH 6.5 containing 50 mM NaCl (100 μl final volume). The substrate (4 μM final concentration) was added after preincubation for 10 min and the kinetics of appearance of the fluorescent signal (RFU) was directly analyzed for 40 min at 28° C. (2.3 min-interval successive measures) by using the fluorimeter reader at 320 nm excitation and 420 nm emission wavelengths. Under these conditions of initial velocity measurement, human NEP-mediated specific hydrolysis of Abz-R-G-F-K-DnpOH was evaluated at 59796±18685 RFU/min/μg rhNEP, n=4 independent determinations.

In addition, the intramolecularly quenched fluorogenic peptide, Mca-BK2 (10 μM), was submitted to hydrolysis by 5 ng rhNEP under the same experimental conditions as those described behind. Under these conditions the hNEP-enzyme acted upon Mca-R-P-P-G-F-S-A-F-K-(Dnp)-OH mainly as a carboxydipeptidase preferentially cleaving A-F bond but also as an endopeptidase cleaving the G-F bond. Under conditions of initial velocity measurement, human NEP-mediated specific hydrolysis of Mca-BK2 was evaluated at 139263±19780 RFU/min/μg rhNEP, n=2 independent determinations.

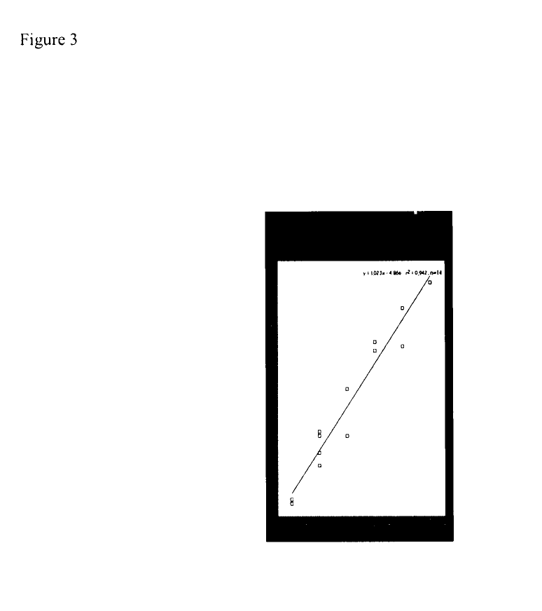

Under these experimental conditions, the pyroglutamate-1 native form of Opiorphin QRFSR (SEQ ID NO: 1)-peptide prevented in a concentration dependent manner the Abz-R-G-F-K-DnpOH cleavage mediated by the rhNEP (r²=0.94, n=14 determination points, FIG. 3).

C. Measurement of AP-N-ectopeptidase activity using Ala-Mca substrate. Using black half-area 96 well microplate the standard reaction consisted of enzyme (4 ng) in 100 mM Tris-HCl pH 7.0 (100 μl final volume). The Ala-Mca substrate (25 μM final concentration) was added after preincubation for 10 min at 28° C. and the kinetics of appearance of the signal was monitored for 40 min at 28° C. by using the fluorimeter reader at 380 nm excitation and 460 nm emission wavelengths. The intensity of the signal was directly proportional to the quantity of metabolites formed during the 10-40 min time-period of the reaction. Under these conditions of initial velocity measurement, the human AP-N-mediated aminoproteolysis of Ala-Mca was directly calculated (from the slope: AP-N activity in absence of inhibitor in function of incubation time) as 147042±44657 RFU/min/μg rhAP-N, n=3 independent determinations.

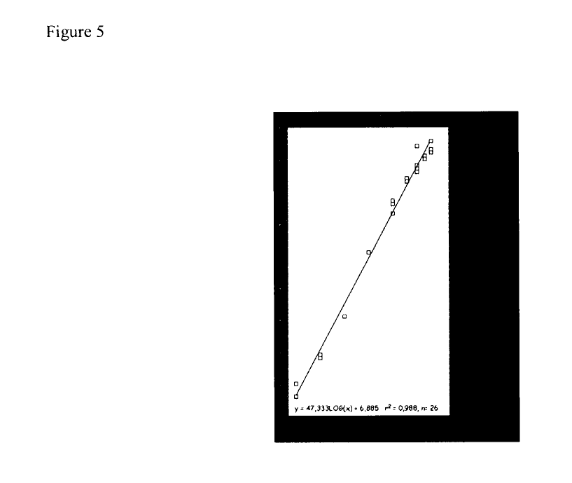

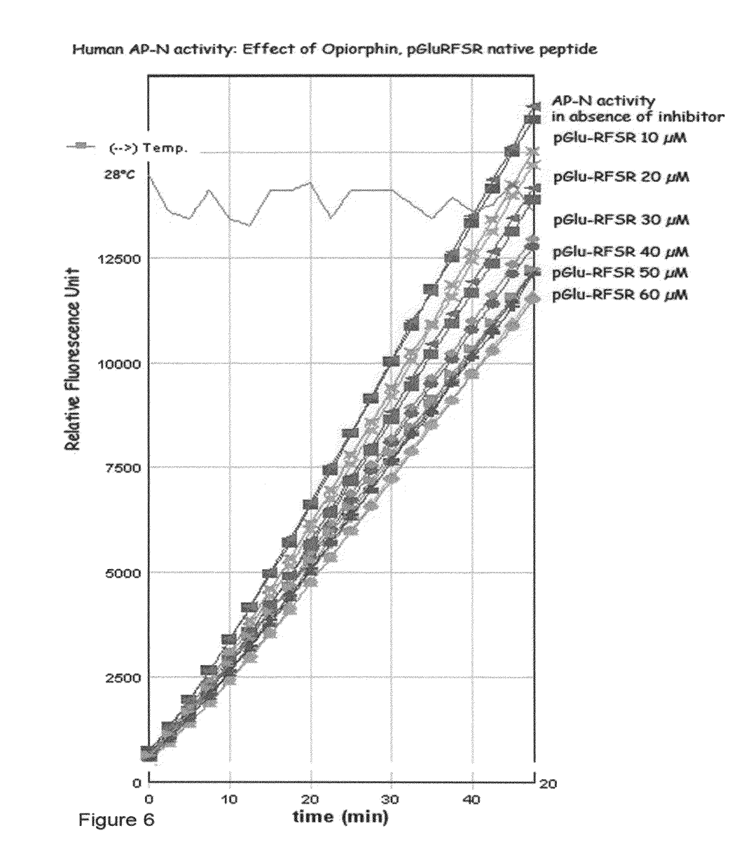

The Glutamine 1 native form of Opiorphin QRFSR (SEQ ID NO: 1)-peptide prevented in a concentration dependent manner the Ala-AMC cleavage mediated by the rhAP-N (r²=0.99, n=26 determination points, FIG. 5). Under the same experimental conditions the inhibitory potency of the pyroglutamate¹ native form of Opiorphin-peptide appeared weak (FIG. 6). This result indicates that the inhibitory potency of Opiorphin towards AP-N requires the amine side-chain of the glutamine-1 of QRFSR (SEQ ID NO: 1)-peptide. This specific requirement was not observed for the inhibitory interaction between Opiorphin and hNEP. On the contrary, it appeared that one of the important group of the QRFSR (SEQ ID NO: 1)-peptide for its inhibitory potency towards hNEP activity is the free carboxyl terminus. Indeed, the QRFSR (SEQ ID NO: 1)-CONH₂ peptide displayed less NEP-inhibitory potency than the QRFSR (SEQ ID NO: 1)-COOH native peptide.

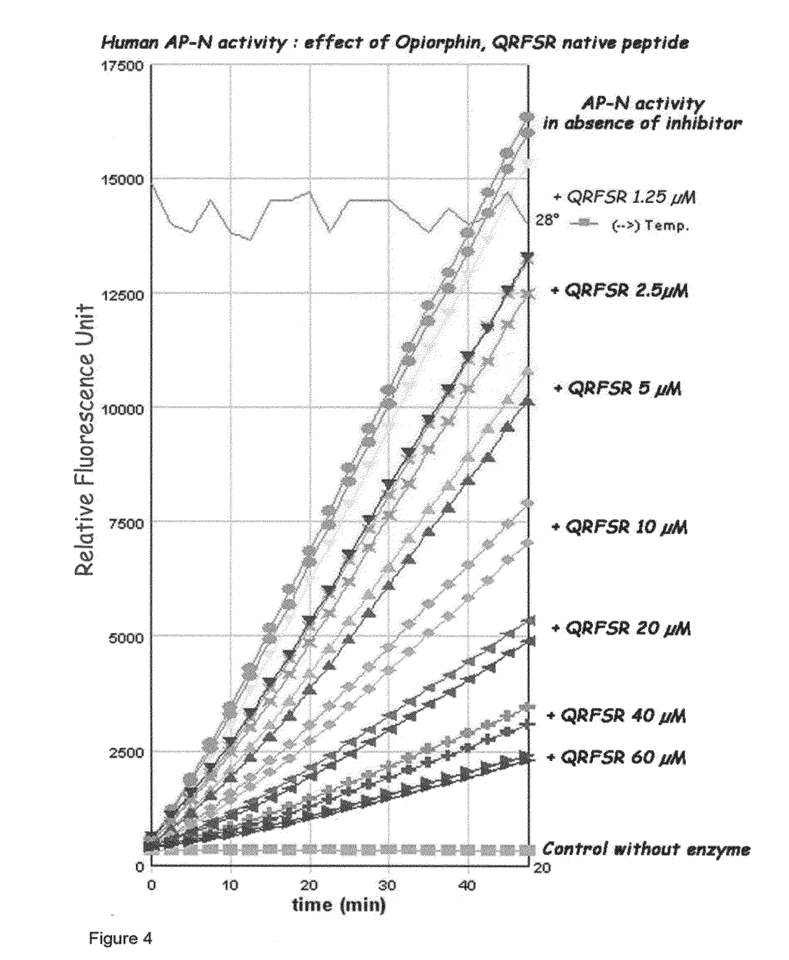

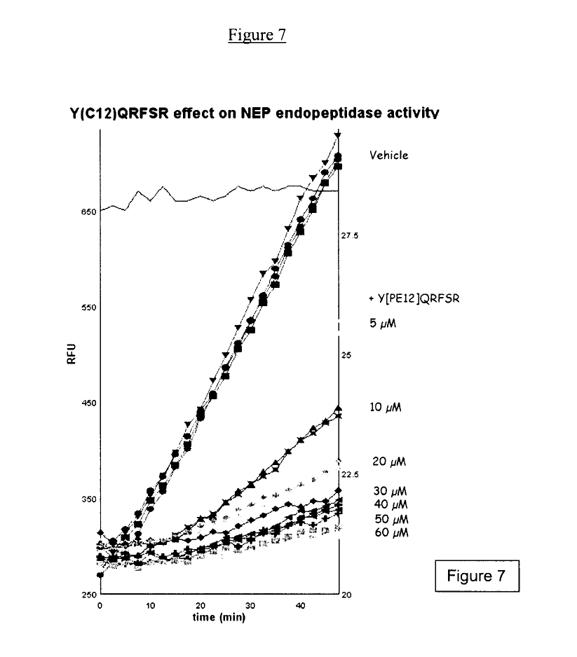

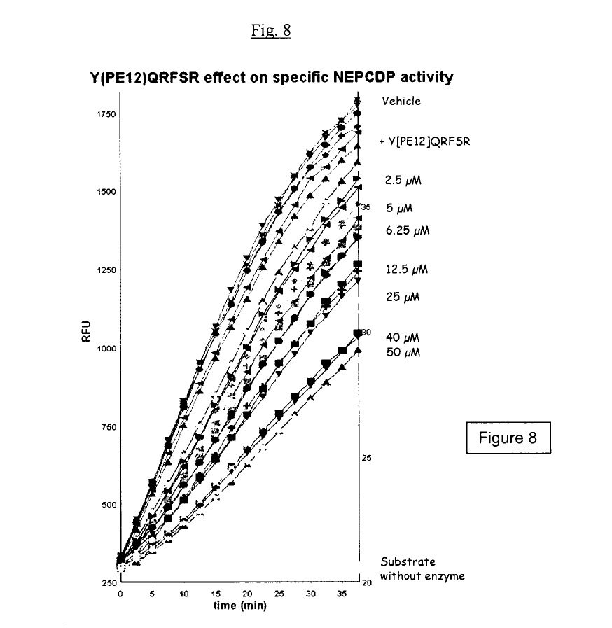

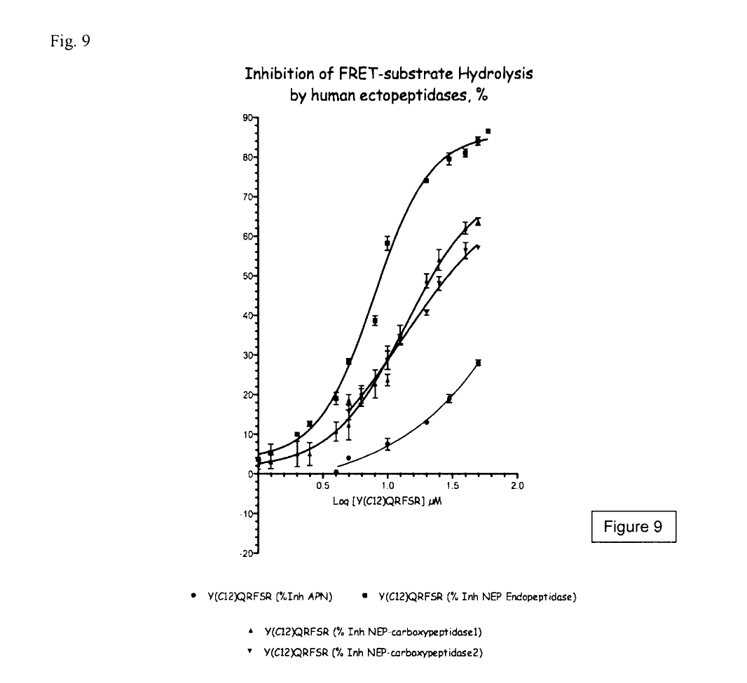

Table 1 below summarizes data appearing in FIGS. 1-9.

TABLE 1

| | | Enzyme acting on | | |
|---|---|---|---|---|
| Figure | Substrate | substrate | Compound | Action |
| 1 | Abz-dRGL-EDDnp | hNEP endopeptidase | QRFSR | Inhibits substrate |
| | | | (SEQ ID NO: 1) | breakdown |
| 2 | Abz-dRGL-EDDnp | hNEP endopeptidase | QRFSR | Inhibits substrate |
| | | | (SEQ ID NO: 1) | breakdown |
| 3 | Abz-R-G-F-K- | hNEP | pGluRFSR | Inhibits substrate |
| | DnpOH | carboxydipeptidase | | breakdown |
| 4 | Ala-AMC | hAP-N ectopeptidase | QRFSR | Inhibits substrate |
| | | | (SEQ ID NO: 1) | breakdown |
| 5 | Ala-AMC | hAP-N ectopeptidase | QRFSR | Inhibits substrate |
| | | | (SEQ ID NO: 1) | breakdown |
| 6 | Ala-AMC | hAP-N ectopeptidase | pGluRFSR | Inhibits substrate |
| | | | | breakdown |
| 7 | Abz-dRGL-EDDnp | hNEP endopeptidase | Y[C12PE]QRFSR | Inhibits substrate |
| (new 1) | | | | breakdown |
| 8 | Abz-R-G-F-K- | hNEP | Y[C12PE]QRFSR | Inhibits substrate |
| (new 2) | DnpOH | carboxydipeptidase | | breakdown |
| 9 | Ala-AMC | hAP-N ectopeptidase | Y[C12PE]QRFSR | Inhibits substrate |
| (new 3) | | | | breakdown |
| 9 ▪ | Abz-dRGL-EDDnp | hNEP endopeptidase | Y[C12PE]QRFSR | Inhibits substrate |
| (new 3) | | | | breakdown |
| 9 ▴ | Mca-BK2 | hNEP | Y[C12PE]QRFSR | Inhibits substrate |
| (new 3) | | carboxydipeptidase 1 | | |
| 9 ▾ | Abz-R-G-F-K- | hNEP | Y[C12PE]QRFSR | Inhibits substrate |
| (new 3) | DnpOH | carboxydipeptidase 2 | | breakdown |

As apparent from these data, substituting the terminal NH₂ group of the Glutamine on Opiorphin by a 12 Carbon-polyethylene spacer plus a Tyrosine residue strongly decreased the inhibitory potency of QRFSR (SEQ ID NO: 1) Opiorphin-peptide towards hAP-N but reinforcing its inhibitory potency towards hNEP-Endopeptidase and -Carboxypeptidase activities.

The inhibitory activity (IC50) of various compounds toward recombinant soluble forms of various hNEP is shown in Table 2 below.

TABLE 2

| | |
|---|---|
| FRET-based | Inhibitory activity (IC50) towards |
| enzyme | recombinant soluble: |

| assay | | hNEP- | hNEP-Carboxy- |
|---|---|---|---|
| Compounds | hAP-N | Endopeptidase | DiPeptidase |
| QRFSR | 10 μM | ~70 μM | ~33 μM |
| (SEQ ID NO: 1) | | | |
| pyroGluRFSR | ≧50 μM | ~50 μM | ~50 μM |
| (SEQ ID NO: 1) | | | |
| Y[PE12]QRFSR | ≧50 μM | ~13-14 μM | ~8 μM |
| (SEQ ID NO: 2) | | | |

In Vivo Assays

BPLP and Opiorphin products and derivatives that have been identified by their agonist or antagonist activities in vitro may be further characterized in vivo, for example, by evaluating nociceptive or other known BPLP and Opiorphin activities after the oral, intravenous, or parenteral administration of these products or derivatives to test subjects. Examples of animal models for nociception are the formalin test (Rougeot et al., Sialorphin, a natural inhibitor of rat membrane-bound neutral endopeptidase that displays analgesic activity. Proc Natl Acad Sci USA 100(14), 8549-8554) and the pin pain test, (Hebert et al. (1999) Physiology & Behavior 67, 99-105). Such assays and tests are specifically incorporated by reference to Rougeot, et al., id. and Herbert, et al., id. cited above.

### Modifications and Other Embodiments

Various modifications and variations of the described methods, BPLP or BPLP maturation products, including Opiorphin, and derivatives thereof and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the medical, biological, chemical or pharmacological arts or related fields are intended to be within the scope of the following claims.

### Incorporation by Reference

Each document, patent application or patent publication cited by or referred to in this disclosure is incorporated by reference in its entirety. Any patent document to which this application claims priority is also incorporated by reference in its entirety. Specifically, priority document U.S. 61/021,988, filed Jan. 18, 2008 is hereby incorporated by reference.

1. An in vitro method for screening compounds for their ability to act as agonists or antagonists of BPLP (basic proline-rich lacrimal protein or PROL1 gene product) or maturation products thereof on hNEP or hAP-N activity, which method comprises:
a) incubating a candidate compound with a soluble hNEP (human neutral ecto-peptidase) or hAP-N (human ecto-aminopeptidase), in the presence of a hNEP or hAP-N substrate;
b) determining the hydrolysis rate of the NEP or AP-N substrate by the hNEP or hAP-N pure soluble enzyme,
wherein an increased hydrolysis rate in the presence of the candidate compound, in comparison with the hydrolysis in the absence of the candidate compound, in initial velocity conditions, is indicative of an antagonist activity; and
wherein a decreased hydrolysis rate in the presence of the candidate compound, in comparison with the hydrolysis in the absence of the candidate compound, is indicative of an agonist activity.

2. The method of claim 1, which screens said compounds for their ability to act as Opiorphin agonists or antagonists, wherein Opiorphin is a maturation product of BPLP.

3. The method of claim 1, which is a method for identifying an agonist of BPLP or a maturation product thereof.

4. The method of claim 1, which is a method for identifying an antagonist of BPLP or a maturation product thereof.

5. The method of claim 1, wherein a) is conducted in the presence of a NEP substrate.

6. The method of claim 1, wherein a) is conducted in the presence of an AP-N substrate.

7. The method of claim 1, wherein the substrate is specific for NEP-endopeptidase activity.

8. The method of claim 1, wherein the substrate is Abz-dR-G-L-EDDnp FRET-peptide.

9. The method of claim 1, wherein the substrate is specific for NEP-carboxydipeptidase activity.

10. The method of claim 1, wherein the substrate is Abz-R-G-F-K-DnpOH FRET-peptide.

11. The method of claim 1, wherein the substrate is Mca-R-P-P-G-F-S-A-F-K-(Dnp)-OH FRET-peptide (Mca-BK2).

12. The method of claim 1, wherein the substrate is specific for aminopeptidase activity.

13. The method of claim 1, wherein the substrate is L-alanine-Mca (Ala-Mca).

14. The method of claim 1, wherein the substrate is a fluorophore-peptide.

15. The method of claim 1, wherein said method is a real-time fluorescence monitoring microplate adapted fluorimetric assay.


SEQUENCE LISTING (3 sequences)

SEQ ID NO: 1 - protein, 5 aa, Homo sapiens
  MISC_FEATURE (1)..(1): Glutamine or pyro-glutamic acid
QRFSR

SEQ ID NO: 2 - protein, 6 aa, Artificial
  synthetic
XQRFSR

SEQ ID NO: 3 - protein, 6 aa, Artificial
  Synthetic
XXRFSR